(19) United States
(12) Patent Application Publication
(10) Pub. No.: US 2016/0128631 A1
(43) Pub. Date: May 12, 2016

(54) VAPORIZATION HEAT LOSS MEASURING APPARATUS, METABOLIC EXPENDITURE MEASURING APPARATUS AND METHOD OF MEASURING VAPORIZATION HEAT LOSS

(71) Applicant: SEIKO EPSON CORPORATION, Tokyo (JP)

(72) Inventor: Akira IKEDA, Chino-shi (JP)

(21) Appl. No.: 14/929,756

(22) Filed: Nov. 2, 2015

(30) Foreign Application Priority Data
Nov. 10, 2014 (JP) 2014-228076

(51) Int. Cl. A61B 5/00

(52) U.S. Cl. CPC A61B 5/4866; A61B 5/4266; A61B 5/7278; A61B 2562/029; A61B 2560/0252; A61B 2562/0271

(57) ABSTRACT

A vaporization heat loss measuring apparatus measures humidity and a temperature of an atmosphere of a surrounding environment using an environmental humidity measuring part and an environmental temperature measuring part. Further, the apparatus measures humidity and a temperature of an atmosphere near a surface of a measuring object using an object humidity measuring part and an object temperature measuring part. Further, the apparatus calculates a vaporization heat loss from the surface of the measuring object using the environmental humidity, the environmental temperature, the object humidity, and the object temperature. Furthermore, the apparatus calculates a non-evaporative heat loss based on a measurement result of a heat flow measuring part, and calculates a metabolic expenditure using the vaporization heat loss and the non-evaporative heat loss.

### BACKGROUND

1. Technical Field

The present invention relates to a vaporization heat loss measuring apparatus etc.

2. Related Art

The temperature of a human body is maintained by heat released from the human body. The metabolism for maintaining the body temperature is called a basal metabolism. That is, a metabolic expenditure may be known by a measurement of the heat released from the human body.

For example, Patent Document 1 (JP-A-2011-120917) discloses a technology of obtaining physiological information or context information from a wearer's body. For example, the document discloses a method of obtaining calorie consumption and a basal metabolic rate from a heart rate, a pulse rate, a respiratory rate, heat flow, and oxygen consumption.

Patent Document 1 discloses a configuration of sensing an amount of perspiration by a measurement of an electrical skin resistance, but does not disclose a technology of obtaining an evaporative heat loss, i.e., a vaporization heat loss. An amount of the vaporization of the perspiration fluid depends on the environmental humidity, and accordingly, in an exercise and a hot environment with profuse perspiration, it is impossible to accurately measure the amount of vaporization heat due to perspiration from the amount of perspiration.

Note that the same problem can arise not only when the measuring object is a human body but also in measurement of a heat loss due to perspiration of another animal and measurement of a vaporization heat loss in a pipe of a factory, a storage facility, or the like.

### SUMMARY

An advantage of some aspects of the invention is to provide a technology of accurately measuring a vaporization heat loss (evaporative heat loss) from a measuring object. Another advantage of some aspects of the invention is to provide a technology of measuring a more accurate metabolic expenditure based on thus obtained vaporization heat loss.

A first aspect of the invention is directed to a vaporization heat loss measuring apparatus including a first temperature sensor that measures a first temperature as a temperature near a surface of a measuring object, a first humidity sensor that measures first humidity as humidity near the surface of the measuring object, a second temperature sensor that measures a second temperature as a temperature of an environment, a second humidity sensor that measures second humidity as humidity of the environment, and a vaporization heat loss calculation part that calculates a vaporization heat loss of the measuring object using the first temperature, the first humidity, the second temperature, and the second humidity.

A second aspect of the invention is directed to the vaporization heat loss measuring apparatus according to the first aspect of the invention, in which the vaporization heat loss calculation part calculates first saturated water vapor pressure as saturated water vapor pressure near the surface of the measuring object based on the first temperature, calculates second saturated water vapor pressure as saturated water vapor pressure of the environment based on the second temperature, and calculates the vaporization heat loss based on a ratio between the first saturated water vapor pressure and the first humidity and a ratio between the second saturated water vapor pressure and the second humidity.

A third aspect of the invention is directed to a vaporization heat loss measuring apparatus including a first saturated water vapor pressure measuring part that measures first saturated water vapor pressure as saturated water vapor pressure near a surface of a measuring object, a second saturated water vapor pressure measuring part that measures second saturated water vapor pressure as saturated water vapor pressure of an environment, and a vaporization heat loss calculation part that calculates a vaporization heat loss of the measuring object using the first saturated water vapor pressure and the second saturated water vapor pressure.

A fourth aspect of the invention is directed to a vaporization heat loss measuring apparatus including a first humidity sensor that measures first humidity as humidity near a surface of a measuring object, a second humidity sensor that measures second humidity as humidity of an environment, and a vaporization heat loss calculation part that calculates a vaporization heat loss of the measuring object using the first humidity and the second humidity.

In all of the first to fourth aspects of the invention, the evaporative heat loss, i.e., the vaporization heat loss from the measuring object to the surrounding atmosphere may be accurately measured.

A fifth aspect of the invention is directed to a metabolic expenditure measuring apparatus including the vaporization heat loss measuring apparatus according to any of the first to fourth aspects of the invention, a heat flow measuring part that measures a heat flow from the surface of the measuring object to the environment, a non-evaporative heat loss calculation part that calculates a non-evaporative heat loss from the measuring object based on the heat flow, and a metabolic expenditure calculation part that calculates a metabolic expenditure of the measuring object using the vaporization heat loss measured by the vaporization heat loss measuring apparatus and the non-evaporative heat loss.

According to the fifth aspect of the invention, the metabolic expenditure may be calculated using the accurate vaporization heat loss obtained from any one of the first to fourth aspects of the invention.

A sixth aspect of the invention is directed to a method of measuring a vaporization heat loss including measuring a first temperature as a temperature near a surface of a measuring object, measuring first humidity as humidity near the surface of the measuring object, measuring a second temperature as a temperature of an environment, measuring second humidity as humidity of the environment, and calculating a vaporization heat loss of the measuring object using the first temperature, the first humidity, the second temperature, and the second humidity.

According to the sixth aspect of the invention, the same advantages as those of the first aspect of the invention may be obtained.

A seventh aspect of the invention is directed to a method of measuring a vaporization heat loss including measuring first saturated water vapor pressure as saturated water vapor pressure near a surface of a measuring object, measuring second saturated water vapor pressure as saturated water vapor pressure of an environment, and calculating a vaporization heat loss of the measuring object using the first saturated water vapor pressure and the second saturated water vapor pressure.

According to the seventh aspect of the invention, the same advantages as those of the third aspect of the invention may be obtained.

An eighth aspect of the invention is directed to a method of measuring a vaporization heat loss including measuring first humidity as humidity near a surface of a measuring object, measuring second humidity as humidity of an environment, and calculating a vaporization heat loss of the measuring object using the first humidity and the second humidity.

According to the eighth aspect of the invention, the same advantages as those of the fourth aspect of the invention may be obtained.

### BRIEF DESCRIPTION OF THE DRAWINGS

The invention will be described with reference to the accompanying drawings, wherein like numbers reference like elements.

FIG. 1 shows a configuration example of a vaporization heat loss measuring apparatus and metabolic expenditure measuring apparatus in the first embodiment.

FIG. 2 is a functional block diagram showing a function configuration example of the vaporization heat loss measuring apparatus and metabolic expenditure measuring apparatus of the first embodiment.

FIG. 3 is a flowchart for explanation of a flow of processing relating to a vaporization heat loss measurement by the vaporization heat loss measuring apparatus and metabolic expenditure measuring apparatus of the first embodiment.

FIG. 4 shows a configuration example of a vaporization heat loss measuring apparatus and metabolic expenditure measuring apparatus in the second embodiment.

FIG. 5 is a functional block diagram showing a function configuration example of the vaporization heat loss measuring apparatus and metabolic expenditure measuring apparatus of the second embodiment.

FIG. 6 is a flowchart for explanation of a flow of processing relating to a measurement by the vaporization heat loss measuring apparatus and metabolic expenditure measuring apparatus of the second embodiment.

### DESCRIPTION OF EXEMPLARY EMBODIMENTS

### First Embodiment

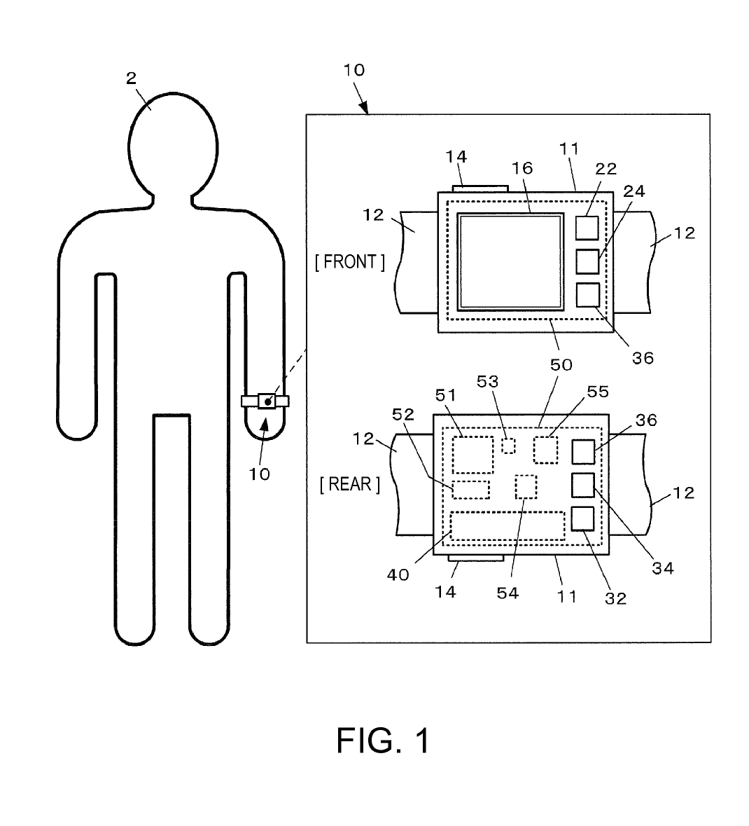

FIG. 1 shows a configuration example of a vaporization heat loss measuring apparatus and metabolic expenditure measuring apparatus (hereinafter, simply referred to as “vaporization heat loss measuring apparatus”) in the embodiment.

A vaporization heat loss measuring apparatus 10 of the embodiment is designed to be wearable on a wrist of a measuring object 2 (a human body in the embodiment). Obviously, the part wearing the apparatus is not limited to the wrist, but may be appropriately changed to a neck part, an upper arm part, an ankle, a part around a chest, a part around a waist, or the like. Further, the measuring object may be another than the human such as an animal.

The vaporization heat loss measuring apparatus 10 is a computer with a sensor for calculation of a vaporization heat loss and a metabolic expenditure. On a side part of an apparatus main body 11, a fixing band 12 and an operation switch 14 that can be used for operation input for a main power supply operation or the like, and the rear surface of the apparatus main body 11 is in close contact or contact with a slight gap with the surface of the measuring object 2 (the skin surface of the wrist in the embodiment).

On the front surface (front face: the surface facing the opposite side to the measuring object 2) of the apparatus main body 11, a touch panel 16, an environmental humidity sensor 22, and an environmental temperature sensor 24 are provided.

The environmental humidity sensor 22 is realized by a known humidity sensor and measures the humidity of the atmosphere of the surrounding environment of the measuring object 2. The environmental temperature sensor 24 is realized by a known temperature sensor and measures the temperature of the surrounding environment of the measuring object 2.

On the rear surface (back face: the surface facing the measuring object 2 in wearing) of the apparatus main body 11, an object humidity sensor 32 and an object temperature sensor 34 are provided. The object humidity sensor 32 and the object temperature sensor 34 are sensors that measure the humidity and the temperature of the atmosphere near the surface of the measuring object 2, respectively. Both may be realized by known sensors.

Further, a heat flow sensor 36 is provided to penetrate from the rear surface to the front surface of the apparatus main body 11. The heat flow sensor 36 is a known sensor having a heat transfer material inside and measuring heat flux density from the temperature difference between the back face and the front face of the heat transfer material and the thermal conductivity of the heat transfer material.

The apparatus main body 11 contains a battery 40 and a control board 50 inside.

The control board 50 has a CPU (central processing unit) 51, an IC memory 52, a touch panel controller 53, a wireless communication device 54, and an interface controller 55 mounted thereon. Obviously, other electronic or electric parts may be appropriately mounted.

The touch panel controller 53 includes known electronic parts and integrated circuits in which functions of image display control for the touch panel 16, output control of operation input signals in response to touch operations made on the touch panel 16, etc. are collected.

The wireless communication device 54 includes known electronic parts or wireless communication device for connection to a known wireless communication circuit (e.g. a mobile telephone network, a short-range radio, a wireless LAN, or the like).

The interface controller 55 includes known electronic parts and integrated circuits having functions of signal input/output control between the operation switch 14 and various sensors and the CPU 51.

The CPU 51 reads out and executes programs stored in the IC memory 52 and realizes various functions for integrated control of the operation of the vaporization heat loss measuring apparatus 10.

For example, the following functions:
1) a function of receiving operation input from the operation switch 14 and the touch panel 16;
2) a function of displaying a user interface screen on the touch panel 16;
3) a function of acquiring measurement results from various sensors;
4) a function of calculating the vaporization heat loss (perspiration vaporization heat loss in the embodiment) from the measuring object 2 based on the measurement results;
5) a function calculating the metabolic expenditure;
6) a timing function;
7) a display control function of displaying the calculated vaporization heat loss and metabolic expenditure on the touch panel 16; and
8) a data log function of saving the calculated vaporization heat loss and metabolic expenditure in the IC memory 52
   are realized. Obviously, other functions may be appropriately realized.

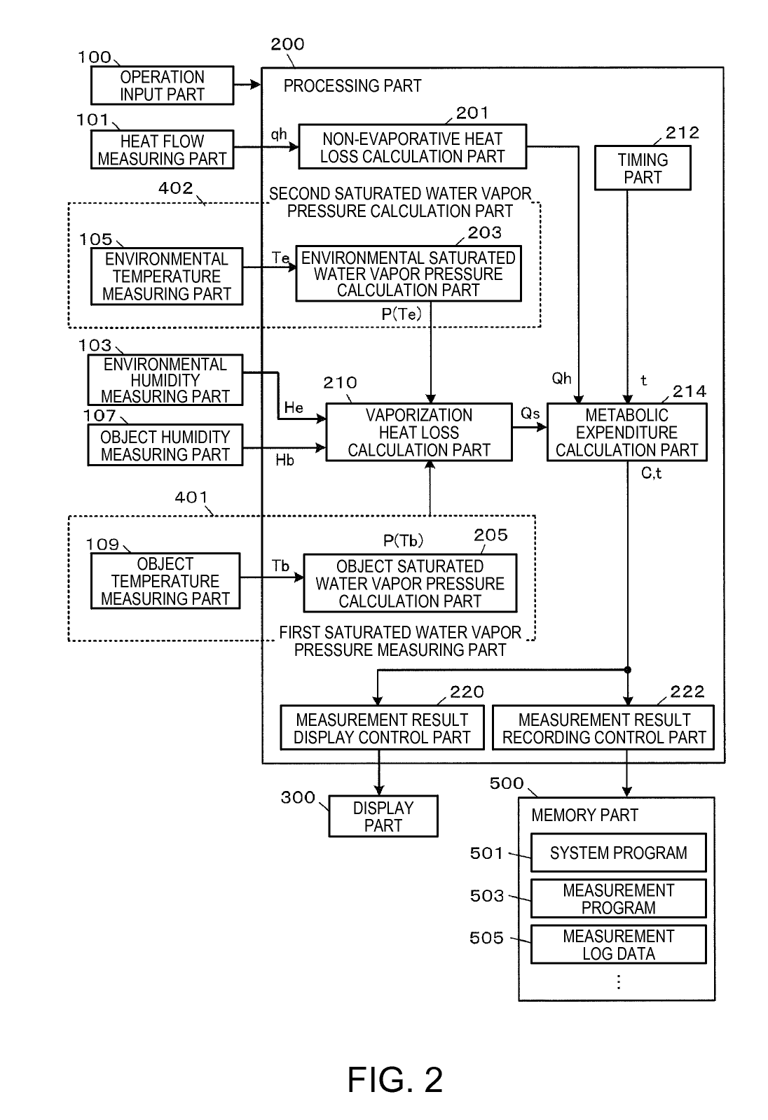

FIG. 2 is a functional block diagram showing a function configuration example of the vaporization heat loss measuring apparatus 10 of the embodiment. The vaporization heat loss measuring apparatus 10 of the embodiment has an operation input part 100, a heat flow measuring part 101, an environmental humidity measuring part 103, an environmental temperature measuring part 105, an object humidity measuring part 107, an object temperature measuring part 109, a processing part 200, a display part 300, and a memory part 500.

The operation input part 100 receives various kinds of operation input by an operator and outputs operation input signals in response to the operation input to the processing part 200. The part may be realized by a button switch, a lever switch, a dial switch, a track pad, a mouse, etc. The operation switch 14 and the touch panel 16 in FIG. 1 correspond to the part.

The heat flow measuring part 101 measures a heat flow due to the heat release from the surface of the measuring object 2 to the environment as a non-evaporative heat loss. In the embodiment, the heat flow sensor 36 in FIG. 1 corresponds to the part, and measures and outputs heat flux density qh to the processing part 200.

The environmental humidity measuring part 103, the object humidity measuring part 107, and the object temperature measuring part 109 measure parameter values for calculation of water vapor pressure.

Specifically, the environmental humidity measuring part 103 measures environmental humidity He (generally, percent, % is used as a unit) as relative humidity of the external environment and outputs a signal in response to the measurement result to the processing part 200. In FIG. 1, the environmental humidity sensor 22 corresponds to the part.

The environmental temperature measuring part 105 measures an environmental temperature Te as a temperature of the external environment and outputs a signal in response to the measurement result to the processing part 200. In FIG. 1, the environmental temperature sensor 24 corresponds to the part.

The object humidity measuring part 107 measures object humidity Hb (generally, percent, % is used as a unit) as relative humidity near the measuring object 2 and outputs a signal in response to the measurement result to the processing part 200. In FIG. 1, the object humidity sensor 32 corresponds to the part.

The object temperature measuring part 109 measures an object temperature Tb as a temperature near the measuring object 2 and outputs a signal in response to the measurement result to the processing part 200. In FIG. 1, the object temperature sensor 34 corresponds to the part.

The processing part 200 realizes various functions of the vaporization heat loss measuring apparatus 10 by arithmetic processing or the like. The control board 50 in FIG. 1 corresponds to the part. In the embodiment, the part includes a non-evaporative heat loss calculation part 201, an environmental saturated water vapor pressure calculation part 203, an object saturated water vapor pressure calculation part 205, a vaporization heat loss calculation part 210, a timing part 212, a metabolic expenditure calculation part 214, a measurement result display control part 220, and a measurement result recording control part 222.

The non-evaporative heat loss calculation part 201 multiplies the heat flux density qh by a predetermined constant of proportionality L to calculate a non-evaporative heat loss Qh.

The environmental saturated water vapor pressure calculation part 203 calculates environmental saturated water vapor pressure P(Te) from a known function that derives a saturated water vapor pressure curve near normal pressure (called a saturated water vapor pressure function e.g. Antoine equation or the like) using the environment temperature Te. In the embodiment, the environmental saturated water vapor pressure calculation part 203 and the environmental temperature measuring part 105 form a second saturated water vapor pressure measuring part 402. The saturated water vapor pressure is uniquely determined as a function of the temperature. Therefore, relationships between the temperature and the saturated water vapor pressure may be stored in a form of a lookup table or the like in the memory part 500, and the environmental saturated water vapor pressure calculation part 203 may obtain the environmental saturated water vapor pressure P(Te) at the environment temperature Te with reference to the lookup table (referred to as “saturated water vapor pressure lookup table”).

The object saturated water vapor pressure calculation part 205 calculates object saturated water vapor pressure P(Tb) near the measuring object surface from the known function that derives the saturated water vapor pressure curve near normal pressure (saturated water vapor pressure function) using the object temperature Tb. In the embodiment, the object saturated water vapor pressure calculation part 205 and the object temperature measuring part 109 form a first saturated water vapor pressure measuring part 401. Similarly, the object saturated water vapor pressure calculation part 205 may obtain the object saturated water vapor pressure P(Tb) at the object temperature Tb with reference to the saturated water vapor pressure lookup table.

The vaporization heat loss calculation part 210 calculates a vaporization heat loss Qs from the surface of the measuring object 2 using the environmental humidity He and the environmental temperature Te, the environmental saturated water vapor pressure P(Te), the object saturated water vapor pressure P(Tb), etc.

The specific calculation method is as follows.

Generally, an amount of water vapor of the atmosphere is proportional to water vapor pressure of the atmosphere. Accordingly, as shown by the formula (1), the environmental humidity He (the relative humidity of the environmental atmosphere surrounding the measuring object 2) is expressed as a ratio of water vapor pressure Pe of the environment (also referred to as “environmental water vapor pressure Pe”) to the environmental saturated water vapor pressure P(Te) at the environmental temperature Te.

$$He = \frac{Pe}{P ( Te )} \qquad Formula ( 1 )$$

Further, similarly, the object humidity Hb as the relative humidity near the surface of the measuring object 2 is expressed as a ratio of water vapor pressure Pb near the measuring object (also referred to as “object water vapor pressure Pb”) to the object saturated water vapor pressure P(Tb) at the object temperature Tb as shown by the formula (2).

$$Hb = \frac{Pb}{P ( Tb )} \qquad Formula ( 2 )$$

Since the degree of vaporization of a liquid attached to a surface such as sweat is proportional to a difference in water vapor pressure, the vaporization heat loss Qs may be obtained as shown in the formula (3) from the formula (1) and the formula (2). Here, K is a constant of proportionality. In this manner, the vaporization heat loss calculation part 210 obtains the vaporization heat loss Qs by multiplying the difference between the environmental water vapor pressure Pe and the object water vapor pressure Pb by the constant of proportionality K. Note that, as shown in the right side of the formula (3), the vaporization heat loss calculation part 210 may obtain the vaporization heat loss Qs by multiplying the difference between a product of the object humidity Hb and the object saturated water vapor pressure P(Tb) and a product of the environmental humidity He and the environmental saturated water vapor pressure P(Te) by the constant of proportionality K.

Qs=K(Pb−Pe)=K(Hb·P(Tb)−He·P(Te)) Formula (3)

The timing part 212 measures a lapse of time. For example, the part is realized by a known timing method using a system clock or a clock.

The metabolic expenditure calculation part 214 calculates a metabolic expenditure C at time t from the non-evaporative heat loss Qh and the vaporization heat loss Qs. Specifically, it is calculated by the formula (4). Note that S and H are constants and obtained in advance by actually measuring a true value of the metabolic expenditure C by an expiratory gas measurement or the like, and concurrently, by using the non-evaporative heat loss Qh and the vaporization heat loss Qs measured by separate attachment of a dedicated apparatus to the human body using a known multivariate analysis such as a multiple regression analysis.

C=S·Qs+H·Qh Formula (4)

However, the constants S and H may be obtained from the formula (5) and the formula (6) with the environmental temperature Te and the object temperature Tb as variables. Note that coefficients M1, M2 and coefficients N1, N2 are obtained to be optimum with respect to the true value of the metabolism measured in advance by the expiratory gas measurement using a known multivariate analysis such as a multiple regression analysis. Then, for example, the coefficients are stored as table data for the environmental temperature Te and the object temperature Tb.

S=M1·Te+N1·Tb Formula (5)

H=M2·Te+N2·Tb Formula (6)

The measurement result display control part 220 performs control for displaying the measurement results of the vaporization heat loss Qs, the non-evaporative heat loss Qh, the metabolic expenditure C, etc. on the display part 300. These numeric values may be displayed in the chronological order or graphical representation.

The display part 300 is image display means and is realized by an image display device such as a flat display panel. In FIG. 1, the touch panel 16 corresponds to the part.

The measurement result recording control part 222 realizes a function as a data logger that stores and records the measurement results in the chronological order in measurement log data 505 of the memory part 500. The function as the data logger may be realized appropriately using a known technology.

The memory part 500 is realized by a storage medium such as an IC memory, a hard disk, or an optical disk, and stores various programs and various kinds of data including data in the calculation process of the processing part 200. In FIG. 1, the IC memory 52 corresponds to the part.

In the embodiment, the part stores a system program 501, a measurement program 503, and the measurement log data 505. In addition, obviously, other data including a counter for timing may be appropriately stored.

The system program 501 is a basic program for allowing the processing part 200 to realize a basic function as a computer.

The measurement program 503 is an application program executed in a state in which the basic program is executed. The processing part 200 is allowed to realize the functions as the non-evaporative heat loss calculation part 201, the environmental saturated water vapor pressure calculation part 203, the object saturated water vapor pressure calculation part 205, the vaporization heat loss calculation part 210, the timing part 212, the metabolic expenditure calculation part 214, the measurement result display control part 220, and the measurement result recording control part 222. When the functions of the processing part 200 are realized by hardware, program elements for realization of the corresponding functions maybe appropriately omitted from the measurement program 503.

The measurement log data 505 stores the measurement results in the chronological order. In the embodiment, measurement numbers automatically assigned in the order of measurements, measurement times and dates, and the measurement results are associated with one another and stored in the chronological order. Obviously, other data may be appropriately associated with each other and stored.

Next, an operation of the vaporization heat loss measuring apparatus 10 will be explained.

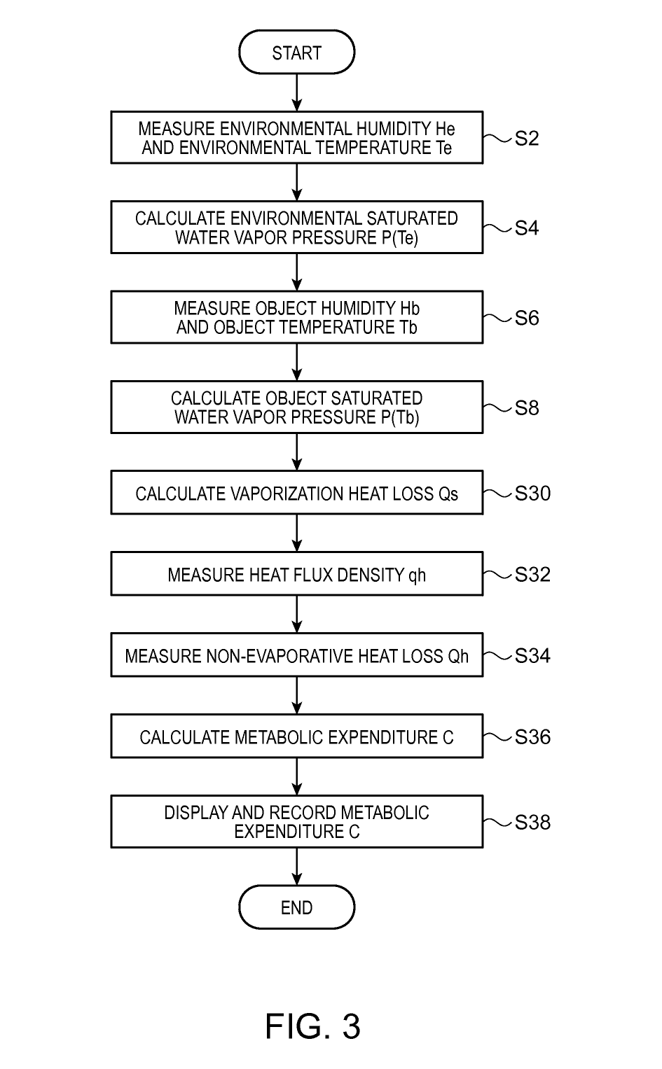

FIG. 3 is a flowchart for explanation of a flow of processing relating to a vaporization heat loss measurement by the vaporization heat loss measuring apparatus 10. When the measurement is continuously performed or intermittently repeated, the flowchart is appropriately repeatedly executed.

The processing part 200 of the vaporization heat loss measuring apparatus 10 first measures the environmental humidity He and the environmental temperature Te (step S2) and calculates the environmental saturated water vapor pressure P(Te) (step S4). Further, the part measures the object humidity Hb and the object temperature Tb (step S6) and calculates the object saturated water vapor pressure P(Tb) near the surface of the measuring object 2 (step S8). The steps S6 and S8 may be executed prior to the steps S2 and S4.

Then, the vaporization heat loss measuring apparatus calculates the vaporization heat loss Qs from the environmental humidity He, the environmental saturated water vapor pressure P(Te), the object humidity Hb, and the object saturated water vapor pressure P(Tb) (step S30).

Then, the vaporization heat loss measuring apparatus 10 measures the heat flux density qh (step S32) and calculates the non-evaporative heat loss Qh (step S34).

Then, the apparatus calculates the metabolic expenditure C using the vaporization heat loss Qs and the non-evaporative heat loss Qh (step S36), displays the calculated metabolic expenditure C on the touch panel 16, and stores it in the IC memory 52 (step S38).

As described above, according to the embodiment, the vaporization heat loss Qs (evaporative heat loss) from the surface of the measuring object 2 may be accurately measured in consideration of the influence by the environmental relative humidity. Further, the more accurate metabolic expenditure C can be obtained based on the obtained vaporization heat loss Qs.

### Second Embodiment

Next, the second embodiment to which the invention is applied will be explained. As below, the differences from the first embodiment will be mainly described, and the same signs are assigned to the same component elements as those of the first embodiment and the overlapping explanation is omitted.

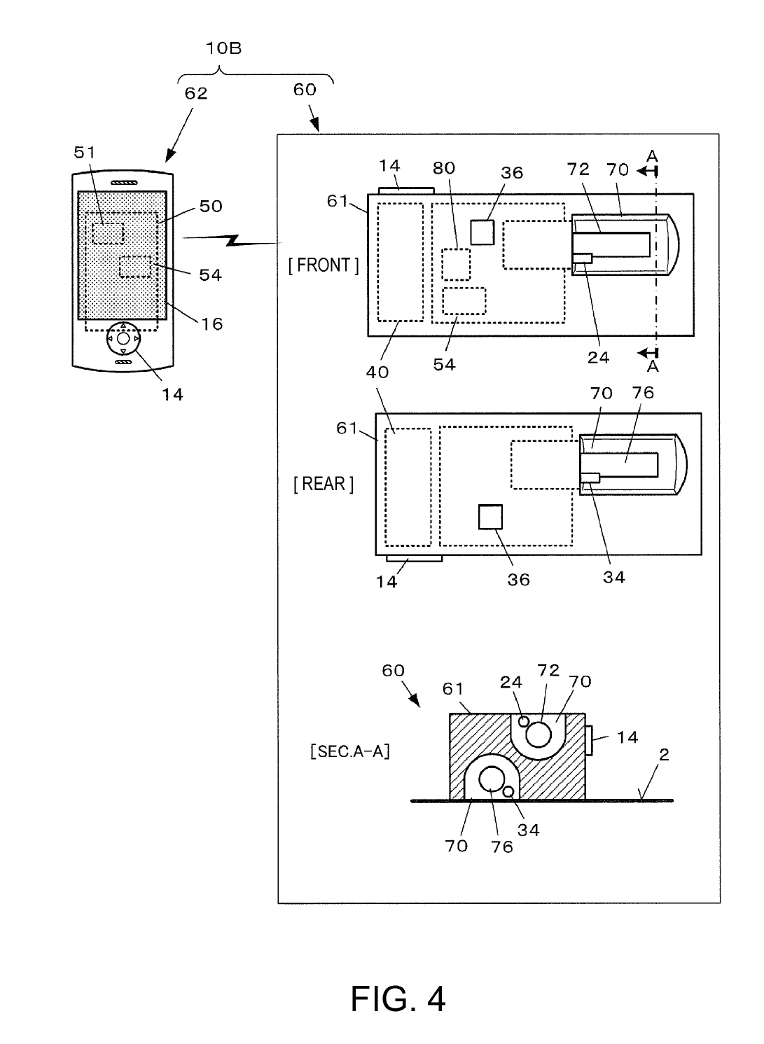

FIG. 4 shows a configuration example of a vaporization heat loss measuring apparatus 10B in the embodiment. The vaporization heat loss measuring apparatus 10B of the embodiment includes a sensor unit 60 to be pressed against the measuring object 2 and a calculation unit 62 connected to the sensor unit 60 to communicate data.

The sensor unit 60 has an operation switch 14 on the side surface of a unit main body 61.

Further, on the front surface of the unit main body 61, a sensor housing space 70 is recessed, and an environmental dew-point sensor 72 that measures the dew point of the environmental atmosphere and an environmental temperature sensor 24 that measures the temperature of the environment are provided within the space. A sensor housing space 70 is also provided on the rear surface of the unit main body 61, and an object dew-point sensor 76 that measures the dew point of the atmosphere near the surface of the measuring object 2 and an object temperature sensor 34 that measures the temperature of the atmosphere near the surface are provided within the space. Furthermore, a heat flow sensor 36 is provided to penetrate the front surface and the rear surface of the unit main body 61. The environmental dew-point sensor 72 and the object dew-point sensor 76 are realized by a known “dew-point sensor”, “dew-point temperature sensor”, “moisture sensor”, or the like.

The sensor unit 60 contains a communication controller 80 and a wireless communication device 54, and the data measured by the environmental dew-point sensor 72, the environmental temperature sensor 24, the object dew-point sensor 76, the object temperature sensor 34, and the heat flow sensor 36 is individually transmitted to the calculation unit 62.

The calculation unit 62 is a computer for arithmetic processing of the data and takes a form of a smartphone in the embodiment, however, may take a form of a notebook personal computer, a tablet personal computer, or a wearable computer of a wristwatch type or the like.

The calculation unit 62 corresponds to apart of the apparatus main body 11 of the first embodiment (see FIG. 1) without the sensors, and calculates the non-evaporative heat loss Qh, the vaporization heat loss Qs, and the metabolic expenditure C based on the data received from the sensor unit 60 and records the data of the metabolic expenditure C.

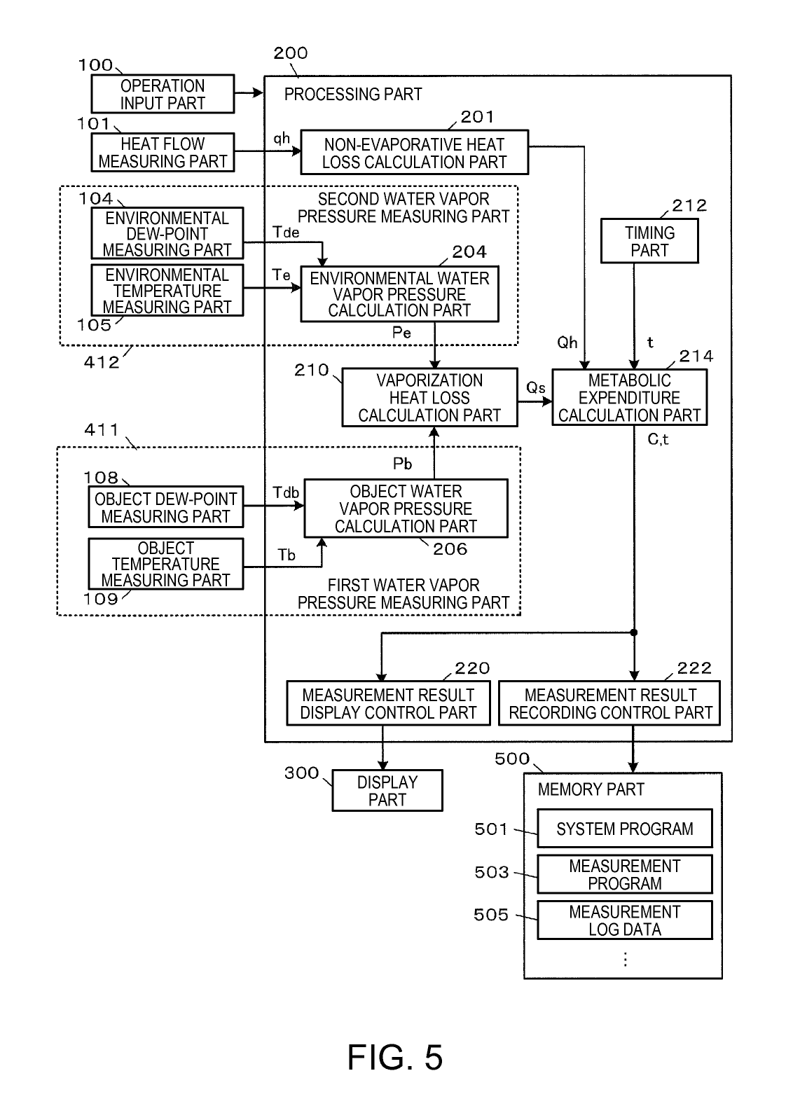

FIG. 5 is a functional block diagram showing a function configuration example of the vaporization heat loss measuring apparatus 10B in the embodiment.

The function configuration in the embodiment is basically the same as that of the first embodiment, however, in place of the environmental humidity measuring part 103 in the first embodiment (see FIG. 2), an environmental dew-point measuring part 104 that measures the dew point Tde of the environmental atmosphere is provided. Further, in place of the environmental saturated water vapor pressure calculation part 203 in the first embodiment (see FIG. 2), an environmental water vapor pressure calculation part 204 that calculates water vapor pressure in the environment (environmental water vapor pressure Pe) is provided.

The dew-point temperature is a temperature at which the water vapor pressure of interest coincides with the saturated water vapor pressure. Therefore, the environmental water vapor pressure calculation part 204 may calculate the environmental water vapor pressure Pe using the environmental dew point Tde. Specifically, the part may calculate the environmental water vapor pressure Pe from the saturated water vapor function and the environmental dew point Tde, or may obtain the environmental water vapor pressure Pe at the environmental dew point Tde with reference to the saturated water vapor pressure lookup table. In FIG. 4, the environmental dew-point sensor 72 corresponds to the environmental dew-point measuring part 104. In the embodiment, the environmental water vapor pressure calculation part 204 and the environmental dew-point measuring part 104 form a second water vapor pressure measuring part 412. Note that, as shown in FIG. 5, the second water vapor pressure measuring part 412 may include the environmental temperature measuring part 105. In this case, the environmental temperature measuring part 105 may also serve as a part of the heat flow measuring part 101.

Further, in the embodiment, in place of the object humidity measuring part 107 in the first embodiment (see FIG. 2), an object dew-point measuring part 108 that measures the dew point Tdb of the atmosphere near the measuring object is provided. In FIG. 4, the object dew-point sensor 76 corresponds to the part.

Furthermore, in place of the object saturated water vapor pressure calculation part 205 in the first embodiment (see FIG. 2), an object water vapor pressure calculation part 206 that calculates the water vapor pressure near the surface of the measuring object 2 (object water vapor pressure Pb) is provided. In the embodiment, the object water vapor pressure calculation part 206 and the object dew-point measuring part 108 form a first water vapor pressure measuring part 411. Note that, as shown in FIG. 5, the first water vapor pressure measuring part 411 may include the object temperature measuring part 109. In this case, the object temperature measuring part 109 may also serve as a part of the heat flow measuring part 101. The first water vapor pressure measuring part 411 calculates the object water vapor pressure Pb using the object dew point Tdb like the second water vapor pressure measuring part 412.

Then, the vaporization heat loss calculation part 210 of the embodiment calculates the vaporization heat loss

Qs based on the environmental water vapor pressure Pe and the object water vapor pressure Pb using the formula (7). Here, K is a constant of proportionality.

Qs=K(Pb−Pe) Formula (7)

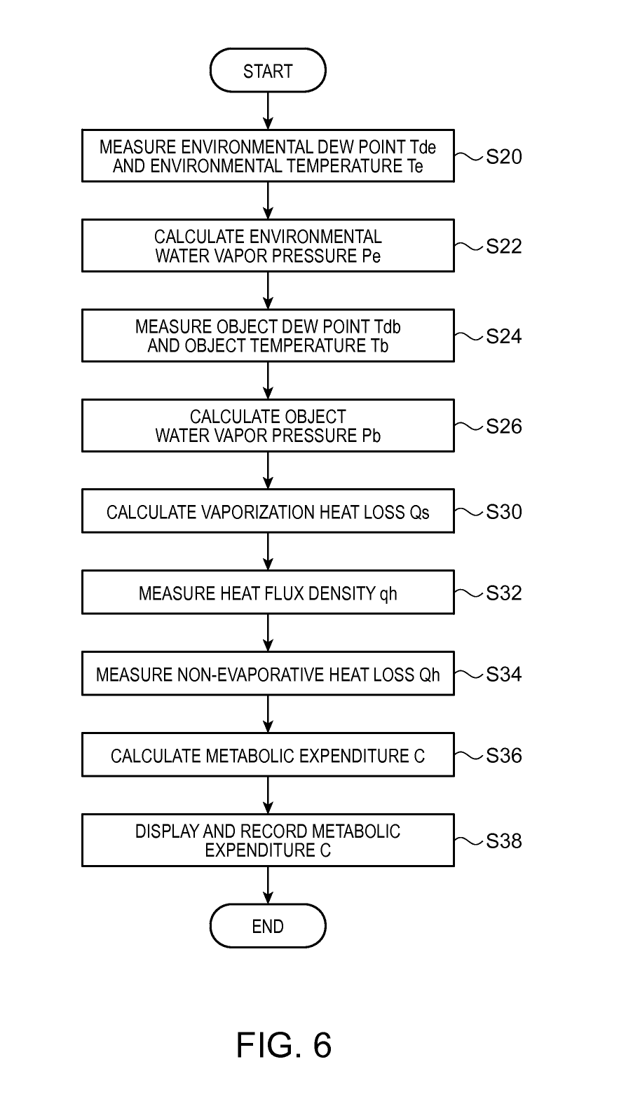

FIG. 6 is a flowchart for explanation of a flow of processing relating to a vaporization heat loss measurement by the vaporization heat loss measuring apparatus 10B of the embodiment.

In place of the steps S2 to S8 in the first embodiment, the processing part 200 of the vaporization heat loss measuring apparatus 10B first measures the environmental dew point Tde and the environmental temperature Te (step S20) and calculates the environmental water vapor pressure Pe (step S22). Further, the part measures the object dew point Tdb and the object temperature Tb (step S24) and calculates the object water vapor pressure Pb near the surface of the measuring object 2 (step S26).

Subsequently, like the first embodiment, the vaporization heat loss measuring apparatus 10B calculates the vaporization heat loss Qs (step S30) and calculates the non-evaporative heat loss Qh based on the heat flux density qh (steps S32, S34). Then, the apparatus calculates the metabolic expenditure C using the vaporization heat loss Qs and the non-evaporative heat loss Qh (step S36), displays the calculated metabolic expenditure C on the touch panel 16 and stores it in the IC memory 52 (step S38).

As described above, according to the embodiment, the same advantages as those of the first embodiment may be obtained.

### MODIFIED EXAMPLE

The embodiments of the invention are not limited to those described above, and addition, omission, changes of the component elements may be appropriately made. For example, in the first embodiment, when the environmental temperature Te and the object temperature Tb are nearly equal, the vaporization heat loss Qs can be calculated by the following formula (8) in place of the formula (3). Here, K′ is a constant of proportionality.

Qs=K′(Hb−He) Formula (8)

The entire disclosure of Japanese Patent Application No. 2014-228076, filed Nov. 10, 2014 is expressly incorporated by reference herein.

What is claimed is:

1. A vaporization heat loss measuring apparatus comprising:
a first temperature sensor that measures a first temperature as a temperature near a surface of a measuring object;
a first humidity sensor that measures first humidity as humidity near the surface of the measuring object;
a second temperature sensor that measures a second temperature as a temperature of an environment;
a second humidity sensor that measures second humidity as humidity of the environment; and
a vaporization heat loss calculation part that calculates a vaporization heat loss of the measuring object using the first temperature, the first humidity, the second temperature, and the second humidity.

2. The vaporization heat loss measuring apparatus according to claim 1, wherein the vaporization heat loss calculation part calculates first saturated water vapor pressure as saturated water vapor pressure near the surface of the measuring object based on the first temperature, calculates second saturated water vapor pressure as saturated water vapor pressure of the environment based on the second temperature, and calculates the vaporization heat loss based on a ratio between the first saturated water vapor pressure and the first humidity and a ratio between the second saturated water vapor pressure and the second humidity.

3. A vaporization heat loss measuring apparatus comprising:
a first saturated water vapor pressure measuring part that measures first saturated water vapor pressure as saturated water vapor pressure near a surface of a measuring object;
a second saturated water vapor pressure measuring part that measures second saturated water vapor pressure as saturated water vapor pressure of an environment; and
a vaporization heat loss calculation part that calculates a vaporization heat loss of the measuring object using the first saturated water vapor pressure and the second saturated water vapor pressure.

4. A vaporization heat loss measuring apparatus comprising:
a first humidity sensor that measures first humidity as humidity near a surface of a measuring object;
a second humidity sensor that measures second humidity as humidity of an environment; and
a vaporization heat loss calculation part that calculates a vaporization heat loss of the measuring object using the first humidity and the second humidity.

5. A metabolic expenditure measuring apparatus comprising:
the vaporization heat loss measuring apparatus according to claim 1;
a heat flow measuring part that measures a heat flow from the surface of the measuring object to the environment;
a non-evaporative heat loss calculation part that calculates a non-evaporative heat loss from the measuring object based on the heat flow; and
a metabolic expenditure calculation part that calculates a metabolic expenditure of the measuring object using the vaporization heat loss measured by the vaporization heat loss measuring apparatus and the non-evaporative heat loss.

6. A metabolic expenditure measuring apparatus comprising:
the vaporization heat loss measuring apparatus according to claim 2;
a heat flow measuring part that measures a heat flow from the surface of the measuring object to the environment;
a non-evaporative heat loss calculation part that calculates a non-evaporative heat loss from the measuring object based on the heat flow; and
a metabolic expenditure calculation part that calculates a metabolic expenditure of the measuring object using the vaporization heat loss measured by the vaporization heat loss measuring apparatus and the non-evaporative heat loss.

7. A metabolic expenditure measuring apparatus comprising:
the vaporization heat loss measuring apparatus according to claim 3;
a heat flow measuring part that measures a heat flow from the surface of the measuring object to the environment;
a non-evaporative heat loss calculation part that calculates a non-evaporative heat loss from the measuring object based on the heat flow; and
a metabolic expenditure calculation part that calculates a metabolic expenditure of the measuring object using the vaporization heat loss measured by the vaporization heat loss measuring apparatus and the non-evaporative heat loss.

8. A metabolic expenditure measuring apparatus comprising:
the vaporization heat loss measuring apparatus according to claim 4;
a heat flow measuring part that measures a heat flow from the surface of the measuring object to the environment;
a non-evaporative heat loss calculation part that calculates a non-evaporative heat loss from the measuring object based on the heat flow; and
a metabolic expenditure calculation part that calculates a metabolic expenditure of the measuring object using the vaporization heat loss measured by the vaporization heat loss measuring apparatus and the non-evaporative heat loss.

9. A metabolic expenditure measuring apparatus comprising:
the vaporization heat loss measuring apparatus according to claim 5;
a heat flow measuring part that measures a heat flow from the surface of the measuring object to the environment;
a non-evaporative heat loss calculation part that calculates a non-evaporative heat loss from the measuring object based on the heat flow; and
a metabolic expenditure calculation part that calculates a metabolic expenditure of the measuring object using the vaporization heat loss measured by the vaporization heat loss measuring apparatus and the non-evaporative heat loss.

10. A method of measuring a vaporization heat loss comprising:
measuring a first temperature as a temperature near a surface of a measuring object;
measuring first humidity as humidity near the surface of the measuring object;
measuring a second temperature as a temperature of an environment;
measuring second humidity as humidity of the environment; and
calculating a vaporization heat loss of the measuring object using the first temperature, the first humidity, the second temperature, and the second humidity.

11. A method of measuring a vaporization heat loss comprising:
measuring first saturated water vapor pressure as saturated water vapor pressure near a surface of a measuring object;
measuring second saturated water vapor pressure as saturated water vapor pressure of an environment; and
calculating a vaporization heat loss of the measuring object using the first saturated water vapor pressure and the second saturated water vapor pressure.

12. A method of measuring a vaporization heat loss comprising:
measuring first humidity as humidity near a surface of a measuring object;
measuring second humidity as humidity of an environment; and
calculating a vaporization heat loss of the measuring object using the first humidity and the second humidity.
